(19) United States
(12) Patent Application Publication
(10) Pub. No.: US 2019/0218299 A1
(43) Pub. Date: Jul. 18, 2019

(54) Methods for Treating Disorders Associated with Fibrosis and Systemic Sclerosis

(71) Applicant: MOMENTA PHARMACEUTICALS, INC., Cambridge, MA (US)

(72) Inventors: Joel PRADINES, Watertown, MA (US); Elma KURTAGIC, Dorchester, MA (US)

(73) Assignee: Momenta Pharmaceuticals, Inc., Cambridge, MA (US)

(21) Appl. No.: 16/302,582

(22) PCT Filed: May 19, 2017
(86) PCT No.: PCT/US2017/033670

§ 371 Date: Nov. 16, 2018

Related U.S. Application Data
(60) Provisional application No. 62/338,826, filed on May 19, 2016.

(51) Int. Cl. C07K 16/28

(52) U.S. Cl. CPC C07K 16/2842; C07K 2317/24

(57) ABSTRACT

Provided herein are methods for treating fibrosis, including diseases or disorders associated with fibrosis, for example systemic sclerosis.

### RELATED APPLICATION

This application claims the benefit under 35 U.S.C. § 119(e) of U.S. provisional application No. 62/338,826, entitled “Methods For Treating Disorders Associated With Fibrosis And Systemic Sclerosis,” filed May 19, 2016 which is incorporated by reference herein in its entirety.

### FIELD OF INVENTION

The present invention relates to methods for treating disorders associated with fibrosis, such as systemic sclerosis, in a subject.

### BACKGROUND OF INVENTION

Systemic sclerosis and, more generally, disorders associated with fibrosis, are characterized by excessive production and accumulation of extracellular matrix proteins (e.g., collagen and/or glycosaminoglycans) which result in hardening of the tissue or organ, and potentially tissue or organ dysfunction. Due to the unknown etiology of some fibrotic disorders and/or the prevalence of fibrosis in a variety of disease pathologies, treatment of these conditions poses a significant challenge.

### SUMMARY OF INVENTION

Aspects of the present disclosure provide methods for treating a disorder associated with fibrosis comprising administering to a subject having a disorder associated with fibrosis a therapeutically effective amount of an agent that modulates the expression and/or activity of any one or more of the proteins set forth in Table 1. In some embodiments, the disorder associated with fibrosis is pulmonary fibrosis, cirrhosis, atrial fibrosis, endomyocardial fibrosis, glial scar, arthrofibrosis, Crohn's disease, Dupuytren's contracture, keloid, mediastinal fibrosis, myelofibrosis, Peyronie's disease, nephrogenic systemic fibrosis, progressive massive fibrosis, retroperitoneal fibrosis, systemic sclerosis, skeletal muscle fibrosis, or adhesive capsulitis.

Other aspects provide methods for treating systemic sclerosis comprising administering to a subject having systemic sclerosis a therapeutically effective amount of an agent that modulates the expression and/or activity of any one or more of the proteins set forth in Table 1. In some embodiments, the systemic sclerosis is limited cutaneous systemic sclerosis or diffuse cutaneous systemic sclerosis.

In some embodiments, the agent inhibits the expression and/or activity of any one or more of the proteins set forth in Table 1. In some embodiments, the agent enhances the expression and/or activity of any one or more of the proteins set forth in Table 1. In some embodiments, the agent is an antibody or fragment thereof, a protein, a fusion protein, a small molecule, or a nucleic acid. In some embodiments, the agent is an antibody that selectively binds to any one or more of the proteins set forth in Table 1. In some embodiments, the agent modulates expression of a nucleic acid encoding any one or more of the proteins set forth in Table 1. In some examples, the agent is selected from any of the agents set forth in Table 3.

In some embodiments, the method further comprises administering one or more additional agents. In some embodiments, the agent is administered with a pharmaceutically acceptable excipient. In some embodiments, the agent is administered in one dose. In other embodiments, the agent is administered in multiple doses. In some embodiments, the agent is administered orally, intraveneously, intraperitoneally, topically, subcutaneously, or by inhalation.

In some embodiments, the subject is a mammalian subject, such as a human subject.

These and other aspects of the invention, as well as various embodiments thereof, will become more apparent in reference to the detailed description of the invention.

Each of the limitations of the invention can encompass various embodiments of the invention. It is, therefore, anticipated that each of the limitations of the invention involving any one element or combination of elements can be included in each aspect of the invention. This invention is not limited in its application to the details of construction and the arrangement of components set forth in the following description. The invention is capable of other embodiments and of being practiced or of being carried out in various ways.

### DETAILED DESCRIPTION

Aspects of the disclosure relate to the identification of drug targets for treatment of fibrosis or systemic sclerosis. The present disclosure provides methods of treating fibrosis by administering an agent that modulates any of the proteins provided in Table 1. Also provided are methods of treating systemic sclerosis by administering an agent that modulates any of the proteins provided in Table 1.

Generally, methods for identifying proteins considered to play a critical role in a disease or disorder can rely on assessment of gene or protein expression data, comparing samples from the disease state with samples from a healthy or normal state. Genes that are most differentially regulated between the diseased state relative to the healthy or normal state are identified and are typically selected as targets for therapeutic intervention, on the basis that the differential expression is an indication that the protein has a significant role in the disease or disorder.

The methods described herein involve targeting genes that are or are not differentially expressed in the disease state relative to a healthy or normal state. Various statistical methods known in the art can be used to classify genes according to whether they are differentially expressed between two different states. As discussed in Example 1, genes can be scored for differential expression between a disease state and a healthy or normal state using various methods known in the art, such as a two-sided and unequal variances t-test. In some embodiments, methods such as the false discovery rate (FDR) method (Benjamini and Hochberg J. Roy. Soc. Ser. B. (1995) 57:289-300) are used on the p-values to correct for multiple testing, and genes are partitioned in two sets. In some embodiments, genes that are differentially expressed are those that have an FDR value less than 0.01, and genes that are non-differentially expressed are those with FDR values greater than 0.01.

According to methods described herein, in some embodiments, genes that are or are not differentially expressed are then assessed for interaction with proteins that are encoded by highly differentially expressed genes, using protein interaction networks. In some embodiments, genes that are themselves differentially expressed or genes that are themselves not differentially expressed but have significant interaction with genes that are highly differentially expressed can be selected as therapeutic targets. Without wishing to be bound by any theory, such genes may encode proteins that are involved in “upstream” processes/pathways involved in the disease or disorder development and pathology, and therefore represent targets for treatment methods. Target proteins identified by methods described further in Example 1 are shown in Table 1. Gene name aliases for genes in Table 1 are provided in Table 2.

TABLE 1
Target Proteins

| Gene ID* | Symbol | Score | Protein Name |
|---|---|---|---|
| 3676 | ITGA4 | 1.40E−12 | integrin, alpha 4 (antigen CD49D, alpha 4 subunit |
| | | | of VLA-4 receptor) |
| 3678 | ITGA5 | 3.20E−11 | integrin, alpha 5 (fibronectin receptor, alpha |
| | | | polypeptide) |
| 3680 | ITGA9 | 3.80E−11 | integrin, alpha 9 |
| 3688 | ITGB1 | 6.90E−11 | integrin, beta 1 (fibronectin receptor, beta |
| | | | polypeptide, antigen CD29 includes MDF2, MSK12) |
| 3672 | ITGA1 | 3.50E−10 | integrin, alpha 1 |
| 22801 | ITGA11 | 3.70E−10 | integrin, alpha 11 |
| 3693 | ITGB5 | 8.50E−10 | integrin, beta 5 |
| 5747 | PTK2 | 8.50E−10 | protein tyrosine kinase 2 |
| 960 | CD44 | 1.40E−09 | CD44 molecule (Indian blood group) |
| 4313 | MMP2 | 1.40E−09 | matrix metallopeptidase 2 (gelatinase A, 72 kDa |
| | | | gelatinase, 72 kDa type IV collagenase) |
| 7450 | VWF | 1.50E−09 | von Willebrand factor |
| 3655 | ITGA6 | 3.00E−09 | integrin, alpha 6 |
| 8516 | ITGA8 | 3.30E−09 | integrin, alpha 8 |
| 7076 | TIMP1 | 3.80E−09 | TIMP metallopeptidase inhibitor 1 |
| 3679 | ITGA7 | 4.30E−09 | integrin, alpha 7 |
| 3674 | ITGA2B | 5.10E−09 | integrin, alpha 2b (platelet glycoprotein IIb of |
| | | | IIb/IIIa complex, antigen CD41) |
| 3695 | ITGB7 | 6.50E−09 | integrin, beta 7 |
| 3696 | ITGB8 | 1.20E−08 | integrin, beta 8 |
| 1634 | DCN | 1.40E−08 | decorin |
| 3685 | ITGAV | 1.70E−08 | integrin, alpha V |
| 7422 | VEGFA | 2.00E−08 | vascular endothelial growth factor A |
| 3673 | ITGA2 | 2.00E−08 | integrin, alpha 2 (CD49B, alpha 2 subunit of VLA- |
| | | | 2 receptor) |
| 2335 | FN1 | 2.20E−08 | fibronectin 1 |
| 2537 | IFI6 | 2.40E−08 | interferon, alpha-inducible protein 6 |
| 6678 | SPARC | 2.40E−08 | secreted protein, acidic, cysteine-rich (osteonectin) |
| 4312 | MMP1 | 3.10E−08 | matrix metallopeptidase 1 (interstitial collagenase) |
| 4318 | MMP9 | 4.20E−08 | matrix metallopeptidase 9 (gelatinase B, 92 kDa |
| | | | gelatinase, 92 kDa type IV collagenase) |
| 2634 | GBP2 | 4.70E−08 | guanylate binding protein 2, interferon-inducible |
| 3675 | ITGA3 | 5.80E−08 | integrin, alpha 3 (antigen CD49C, alpha 3 subunit |
| | | | of VLA-3 receptor) |
| 51447 | IP6K2 | 7.00E−08 | inositol hexakisphosphate kinase 2 |
| 54739 | XAF1 | 8.20E−08 | XIAP associated factor 1 |
| 1462 | VCAN | 1.00E−07 | versican |
| 3430 | IFI35 | 1.70E−07 | interferon-induced protein 35 |
| 7058 | THBS2 | 1.80E−07 | thrombospondin 2 |
| 4314 | MMP3 | 2.00E−07 | matrix metallopeptidase 3 (stromelysin 1, |
| | | | progelatinase) |
| 3691 | ITGB4 | 2.40E−07 | integrin, beta 4 |
| 3394 | IRF8 | 2.50E−07 | interferon regulatory factor 8 |
| 10581 | IFITM2 | 2.80E−07 | interferon induced transmembrane protein 2 |
| 4811 | NID1 | 2.90E−07 | nidogen 1 |
| 3434 | IFIT1 | 3.00E−07 | interferon-induced protein with tetratricopeptide |
| | | | repeats 1 |
| 7040 | TGFB1 | 3.20E−07 | transforming growth factor, beta 1 |
| 7412 | VCAM1 | 4.00E−07 | vascular cell adhesion molecule 1 |
| 10410 | IFITM3 | 4.70E−07 | interferon induced transmembrane protein 3 |
| 3433 | IFIT2 | 5.20E−07 | interferon-induced protein with tetratricopeptide |
| | | | repeats 2 |
| 2199 | FBLN2 | 5.30E−07 | fibulin 2 |
| 3107 | HLA-C | 6.30E−07 | major histocompatibility complex, class I, C |
| 8519 | IFITM1 | 7.40E−07 | interferon induced transmembrane protein 1 |
| 2277 | FIGF | 8.10E−07 | c-fos induced growth factor (vascular endothelial |
| | | | growth factor D) |
| 7045 | TGFBI | 1.10E−06 | transforming growth factor, beta-induced, 68 kDa |
| 3273 | HRG | 1.20E−06 | histidine-rich glycoprotein |
| 7057 | THBS1 | 1.20E−06 | thrombospondin 1 |
| 284 | ANGPT1 | 1.30E−06 | angiopoietin 1 |
| 3664 | IRF6 | 1.70E−06 | interferon regulatory factor 6 |
| 3437 | IFIT3 | 1.80E−06 | interferon-induced protein with tetratricopeptide |
| | | | repeats 3 |
| 7098 | TLR3 | 1.90E−06 | toll-like receptor 3 |
| 5473 | PPBP | 2.00E−06 | pro-platelet basic protein (chemokine (C-X-C |
| | | | motif) ligand 7) |
| 1490 | CTGF | 2.10E−06 | connective tissue growth factor |
| 22915 | MMRN1 | 2.30E−06 | multimerin 1 |
| 9564 | BCAR1 | 3.30E−06 | breast cancer anti-estrogen resistance 1 |
| 3665 | IRF7 | 3.30E−06 | interferon regulatory factor 7 |
| 1003 | CDH5 | 3.50E−06 | cadherin 5, type 2 (vascular endothelium) |
| 103 | ADAR | 3.60E−06 | adenosine deaminase, RNA-specific |
| 5340 | PLG | 3.70E−06 | plasminogen |
| 1675 | CFD | 3.90E−06 | complement factor D (adipsin) |
| 10379 | IRF9 | 4.00E−06 | interferon regulatory factor 9 |
| 5552 | SRGN | 4.00E−06 | serglycin |
| 6041 | RNASEL | 4.10E−06 | ribonuclease L (2′,5′-oligoisoadenylate synthetase- |
| | | | dependent) |
| 3339 | HSPG2 | 4.20E−06 | heparan sulfate proteoglycan 2 |
| 710 | SERPING1 | 4.50E−06 | serpin peptidase inhibitor, clade G (C1 inhibitor), |
| | | | member 1 |
| 5881 | RAC3 | 4.50E−06 | ras-related C3 botulinum toxin substrate 3 (rho |
| | | | family, small GTP binding protein Rac3) |
| 91543 | RSAD2 | 4.70E−06 | radical S-adenosyl methionine domain containing 2 |
| 3684 | ITGAM | 5.00E−06 | integrin, alpha M (complement component 3 |
| | | | receptor 3 subunit) |
| 3106 | HLA-B | 5.00E−06 | major histocompatibility complex, class I, B |
| 6714 | SRC | 5.10E−06 | v-src avian sarcoma (Schmidt-Ruppin A-2) viral |
| | | | oncogene homolog |
| 81 | ACTN4 | 5.20E−06 | actinin, alpha 4 |
| 7094 | TLN1 | 5.20E−06 | talin 1 |
| 3687 | ITGAX | 5.30E−06 | integrin, alpha X (complement component 3 |
| | | | receptor 4 subunit) |
| 87 | ACTN1 | 5.70E−06 | actinin, alpha 1 |
| 3669 | ISG20 | 5.90E−06 | interferon stimulated exonuclease gene 20 kDa |
| 7448 | VTN | 6.10E−06 | vitronectin |
| 3481 | IGF2 | 6.20E−06 | insulin-like growth factor 2 (somatomedin A) |
| 51284 | TLR7 | 6.60E−06 | toll-like receptor 7 |
| 6773 | STAT2 | 6.90E−06 | signal transducer and activator of transcription 2, |
| | | | 113 kDa |
| 5829 | PXN | 7.00E−06 | paxillin |
| 3662 | IRF4 | 7.20E−06 | interferon regulatory factor 4 |
| 4322 | MMP13 | 7.80E−06 | matrix metallopeptidase 13 (collagenase 3) |
| 7114 | TMSB4X | 9.80E−06 | thymosin beta 4, X-linked |
| 7423 | VEGFB | 9.80E−06 | vascular endothelial growth factor B |
| 7043 | TGFB3 | 1.00E−05 | transforming growth factor, beta 3 |
| 6385 | SDC4 | 1.00E−05 | syndecan 4 |
| 3383 | ICAM1 | 1.10E−05 | intercellular adhesion molecule 1 |
| 22918 | CD93 | 1.10E−05 | CD93 molecule |
| 2200 | FBN1 | 1.20E−05 | fibrillin 1 |
| 2247 | FGF2 | 1.20E−05 | fibroblast growth factor 2 (basic) |
| 5008 | OSM | 1.30E−05 | oncostatin M |
| 4938 | OAS1 | 1.50E−05 | 2′-5′-oligoadenylate synthetase 1, 40/46 kDa |
| 3661 | IRF3 | 1.60E−05 | interferon regulatory factor 3 |
| 3565 | IL4 | 1.70E−05 | interleukin 4 |
| 4317 | MMP8 | 1.90E−05 | matrix metallopeptidase 8 (neutrophil collagenase) |
| 57180 | ACTR3B | 1.90E−05 | ARP3 actin-related protein 3 homolog B (yeast) |
| 5155 | PDGFB | 1.90E−05 | platelet-derived growth factor beta polypeptide |
| 7042 | TGFB2 | 1.90E−05 | transforming growth factor, beta 2 |
| 3105 | HLA-A | 1.90E−05 | major histocompatibility complex, class I, A |
| 6696 | SPP1 | 2.00E−05 | secreted phosphoprotein 1 |
| 4600 | MX2 | 2.00E−05 | myxovirus (influenza virus) resistance 2 (mouse) |
| 4323 | MMP14 | 2.10E−05 | matrix metallopeptidase 14 (membrane-inserted) |
| 4599 | MX1 | 2.20E−05 | myxovirus (influenza virus) resistance 1, |
| | | | interferon-inducible protein p78 (mouse) |
| 5196 | PF4 | 2.30E−05 | platelet factor 4 |
| 3479 | IGF1 | 2.40E−05 | insulin-like growth factor 1 (somatomedin C) |
| 706 | TSPO | 2.60E−05 | translocator protein (18 kDa) |
| 351 | APP | 2.70E−05 | amyloid beta (A4) precursor protein |
| 3660 | IRF2 | 3.00E−05 | interferon regulatory factor 2 |
| 3689 | ITGB2 | 3.10E−05 | integrin, beta 2 (complement component 3 receptor |
| | | | 3 and 4 subunit) |
| 1311 | COMP | 3.40E−05 | cartilage oligomeric matrix protein |
| 8638 | OASL | 3.50E−05 | 2′-5′-oligoadenylate synthetase-like |
| 5627 | PROS1 | 4.10E−05 | protein S (alpha) |
| 3663 | IRF5 | 4.20E−05 | interferon regulatory factor 5 |
| 6382 | SDC1 | 4.70E−05 | syndecan 1 |
| 5653 | KLK6 | 4.70E−05 | kallikrein-related peptidase 6 |
| 3026 | HABP2 | 4.70E−05 | hyaluronan binding protein 2 |
| 4147 | MATN2 | 4.90E−05 | matrilin 2 |
| 4940 | OAS3 | 5.20E−05 | 2′-5′-oligoadenylate synthetase 3, 100 kDa |
| 387 | RHOA | 5.20E−05 | ras homolog family member A |
| 10096 | ACTR3 | 5.20E−05 | ARP3 actin-related protein 3 homolog (yeast) |
| 4921 | DDR2 | 5.30E−05 | discoidin domain receptor tyrosine kinase 2 |
| 10561 | IFI44 | 5.50E−05 | interferon-induced protein 44 |
| 88 | ACTN2 | 5.60E−05 | actinin, alpha 2 |
| 2 | A2M | 5.80E−05 | alpha-2-macroglobulin |
| 176 | ACAN | 7.10E−05 | aggrecan |
| 3627 | CXCL10 | 7.10E−05 | chemokine (C-X-C motif) ligand 10 |
| 1958 | EGR1 | 7.20E−05 | early growth response 1 |
| 3659 | IRF1 | 7.30E−05 | interferon regulatory factor 1 |
| 1191 | CLU | 7.60E−05 | clusterin |
| 2633 | GBP1 | 8.20E−05 | guanylate binding protein 1, interferon-inducible |
| 2153 | F5 | 8.30E−05 | coagulation factor V (proaccelerin, labile factor) |
| 6387 | CXCL12 | 8.50E−05 | chemokine (C-X-C motif) ligand 12 |
| 1991 | ELANE | 8.60E−05 | elastase, neutrophil expressed |
| 80162 | ATHL1 | 9.20E−05 | ATH1, acid trehalase-like 1 (yeast) |
| 2534 | FYN | 9.70E−05 | FYN oncogene related to SRC, FGR, YES |
| 4939 | OAS2 | 9.80E−05 | 2′-5′-oligoadenylate synthetase 2, 69/71 kDa |
| 325 | APCS | 1.00E−04 | amyloid P component, serum |
| 998 | CDC42 | 1.00E−04 | cell division cycle 42 |
| 4035 | LRP1 | 1.10E−04 | low density lipoprotein receptor-related protein 1 |
| 7124 | TNF | 1.60E−04 | tumor necrosis factor |
| 2162 | F13A1 | 1.90E−04 | coagulation factor XIII, A1 polypeptide |
| 3569 | IL6 | 2.00E−04 | interleukin 6 (interferon, beta 2) |
| 3683 | ITGAL | 2.90E−04 | integrin, alpha L (antigen CD11A (p180), |
| | | | lymphocyte function-associated antigen 1; alpha |
| | | | polypeptide) |
| 11274 | USP18 | 4.20E−04 | ubiquitin specific peptidase 18 |
| 3576 | IL8 | 4.30E−04 | interleukin 8 |
| 1511 | CTSG | 7.00E−04 | cathepsin G |
| 4688 | NCF2 | 1.10E−03 | neutrophil cytosolic factor 2 |
| 24138 | IFIT5 | 1.80E−03 | interferon-induced protein with tetratricopeptide |
| | | | repeats 5 |
| 3071 | NCKAP1L | 2.30E−03 | NCK-associated protein 1-like |
| 7097 | TLR2 | 2.60E−03 | toll-like receptor 2 |
| 9332 | CD163 | 2.70E−03 | CD163 molecule |
| 6404 | SELPLG | 3.60E−03 | selectin P ligand |
| 1794 | DOCK2 | 4.20E−03 | dedicator of cytokinesis 2 |
| 8515 | ITGA10 | 5.00E−03 | integrin, alpha 10 |
| 10437 | IFI30 | 5.20E−03 | interferon, gamma-inducible protein 30 |
| 23586 | DDX58 | 5.90E−03 | DEAD (Asp-Glu-Ala-Asp) box polypeptide 58 |
| 5217 | PFN2 | 2.00E−02 | profilin 2 |
| 10346 | TRIM22 | 3.50E−02 | tripartite motif containing 22 |
| 9938 | ARHGAP25 | 5.60E−02 | Rho GTPase activating protein 25 |
| 716 | C1S | 9.70E−02 | complement component 1, s subcomponent |
| 9672 | SDC3 | 1.10E−01 | syndecan 3 |
| 2207 | FCER1G | 1.60E−01 | Fc fragment of IgE, high affinity I, receptor for; |
| | | | gamma polypeptide |
| 10875 | FGL2 | 1.70E−01 | fibrinogen-like 2 |
| 6279 | S100A8 | 1.80E−01 | S100 calcium binding protein A8 |
| 2217 | FCGRT | 1.90E−01 | Fc fragment of IgG, receptor, transporter, alpha |
| 4332 | MNDA | 1.90E−01 | myeloid cell nuclear differentiation antigen |
| 9056 | SLC7A7 | 2.80E−01 | solute carrier family 7 (amino acid transporter light |
| | | | chain, y + L system), member 7 |
| 1043 | CD52 | 3.30E−01 | CD52 molecule |
| 10261 | IGSF6 | 5.10E−01 | immunoglobulin superfamily, member 6 |

*Gene ID refers to the Entrez Gene identifier

TABLE 2
Gene Aliases

| Gene ID* | Symbol | Score | Alias |
|---|---|---|---|
| 3676 | ITGA4 | 1.40E−12 | CD49D, IA4, ITGA4 |
| 3678 | ITGA5 | 3.20E−11 | CD49e, FNRA, VLA-5, VLA5A, ITGA5 |
| 3680 | ITGA9 | 3.80E−11 | ALPHA-RLC, ITGA4L, RLC, ITGA9 |
| 3688 | ITGB1 | 6.90E−11 | CD29, FNRB, GPIIA, MDF2, MSK12, VLA-BETA, |
| | | | VLAB, ITGB1 |
| 3672 | ITGA1 | 3.50E−10 | CD49a, VLA1, ITGA1 |
| 22801 | ITGA11 | 3.70E−10 | HsT18964, ITGA11 |
| 3693 | ITGB5 | 8.50E−10 | ITGB5 |
| 5747 | PTK2 | 8.50E−10 | FADK, FAK, FAK1, FRNK, PPP1R71, p125FAK, |
| | | | pp125FAK, PTK2 |
| 960 | CD44 | 1.40E−09 | CDW44, CSPG8, ECMR-III, HCELL, HUTCH-I, |
| | | | IN, LHR, MC56, MDU2, MDU3, MIC4, Pgp1, |
| | | | CD44 |
| 4313 | MMP2 | 1.40E−09 | CLG4, CLG4A, MMP-2, MMP-II, MONA, TBE-1, |
| | | | MMP2 |
| 7450 | VWF | 1.50E−09 | F8VWF, VWD, VWF |
| 3655 | ITGA6 | 3.00E−09 | CD49f, ITGA6B, VLA-6, ITGA6 |
| 8516 | ITGA8 | 3.30E−09 | ITGA8 |
| 7076 | TIMP1 | 3.80E−09 | EPO, CLGI, EPA, HCI, TIMP, TIMP1 |
| 3679 | ITGA7 | 4.30E−09 | ITGA7 |
| 3674 | ITGA2B | 5.10E−09 | BDPLT16, BDPLT2, CD41, CD41B, GP2B, GPIIb, |
| | | | GT, GTA, HPA3, PPP1R93, ITGA2B |
| 3695 | ITGB7 | 6.50E−09 | ITGB7 |
| 3696 | ITGB8 | 1.20E−08 | ITGB8 |
| 1634 | DCN | 1.40E−08 | CSCD, DSPG2, PG40, PGII, PGS2, SLRR1B, DCN |
| 3685 | ITGAV | 1.70E−08 | CD51, MSK8, VNRA, VTNR, ITGAV |
| 7422 | VEGFA | 2.00E−08 | MVCD1, VEGF, VPF, VEGFA |
| 3673 | ITGA2 | 2.00E−08 | BR, CD49B, GPIa, HPA-5, VLA-2, VLAA2, |
| | | | ITGA2 |
| 2335 | FN1 | 2.20E−08 | CIG, ED-B, FINC, FN, FNZ, GFND, GFND2, |
| | | | LETS, MSF, FN1 |
| 2537 | IFI6 | 2.40E−08 | 6-16, FAM14C, G1P3, IFI-6-16, IFI616, IFI6 |
| 6678 | SPARC | 2.40E−08 | BM-40, ON, SPARC |
| 4312 | MMP1 | 3.10E−08 | CLGN, CLG, MMP1 |
| 4318 | MMP9 | 4.20E−08 | CLG4B, GELB, MANDP2, MMP-9, MMP9 |
| 2634 | GBP2 | 4.70E−08 | GBP2 |
| 3675 | ITGA3 | 5.80E−08 | CD49C, GAP-B3, GAPB3, ILNEB, MSK18, VCA-2, |
| | | | VL3A, VLA3a, ITGA3 |
| 51447 | IP6K2 | 7.00E−08 | IHPK2, PIUS, IP6K2 |
| 54739 | XAF1 | 8.20E−08 | BIRC4BP, HSXIAPAF1, XIAPAF1, XAF1 |
| 1462 | VCAN | 1.00E−07 | CSPG2, ERVR, GHAP, PG-M, WGN, WGN1, |
| | | | VCAN |
| 3430 | IFI35 | 1.70E−07 | IFP35, IFI35 |
| 7058 | THBS2 | 1.80E−07 | TSP2, THBS2 |
| 4314 | MMP3 | 2.00E−07 | CHDS6, MMP-3, SL-1, STMY, STMY1, STR1, |
| | | | MMP3 |
| 3691 | ITGB4 | 2.40E−07 | CD104, ITGB4 |
| 3394 | IRF8 | 2.50E−07 | H-ICSBP, ICSBP, ICSBP1, IMD32A, IMD32B, |
| | | | IRF-8, IRF8 |
| 10581 | IFITM2 | 2.80E−07 | 1-8D, DSPA2c, IFITM2 |
| 4811 | NID1 | 2.90E−07 | NID, NID1 |
| 3434 | IFIT1 | 3.00E−07 | C56, G10P1, IFI-56, IFI-56K, IFI56, IFIT-1, |
| | | | IFNAI1, ISG56, P56, RNM561, IFIT1 |
| 7040 | TGFB1 | 3.20E−07 | LAP, CED, DPD1, TGFB, TGFbeta, TGFB1 |
| 7412 | VCAM1 | 4.00E−07 | CD106, INCAM-100, VCAM1 |
| 10410 | IFITM3 | 4.70E−07 | 1-8U, DSPA2b, IP15, IFITM3 |
| 3433 | IFIT2 | 5.20E−07 | P54, G10P2, GARG-39, IFI-54, IFI-54K, IFI54, |
| | | | IFIT-2, ISG-54 K, ISG-54K, ISG54, cig42, IFIT2 |
| 2199 | FBLN2 | 5.30E−07 | FBLN2 |
| 3107 | HLA-C | 6.30E−07 | D6S204, HLA-JY3, HLC-C, PSORS1, HLA-C |
| 8519 | IFITM1 | 7.40E−07 | 9-27, CD225, DSPA2a, IFI17, LEU13, IFITM1 |
| 2277 | FIGF | 8.10E−07 | VEGF-D, VEGFD, FIGF |
| 7045 | TGFBI | 1.10E−06 | BIGH3, CDB1, CDG2, CDGG1, CSD, CSD1, |
| | | | CSD2, CSD3, EBMD, LCD1, TGFBI |
| 3273 | HRG | 1.20E−06 | HRG, HPRG, HRGP, THPH11 |
| 7057 | THBS1 | 1.20E−06 | THBS, THBS-1, TSP, TSP-1, TSP1, THBS1 |
| 284 | ANGPT1 | 1.30E−06 | AGP1, AGPT, ANG1, ANGPT1 |
| 3664 | IRF6 | 1.70E−06 | LPS, OFC6, PIT, PPS, PPS1, VWS, VWS1, IRF6 |
| 3437 | IFIT3 | 1.80E−06 | CIG-49, GARG-49, IFI60, IFIT4, IRG2, ISG60, |
| | | | P60, RIG-G, cig41, IFIT3 |
| 7098 | TLR3 | 1.90E−06 | CD283, IIAE2, TLR3 |
| 5473 | PPBP | 2.00E−06 | PBP, B-TG1, Beta-TG, CTAP-III, CTAP3, |
| | | | CTAPIII, CXCL7, LA-PF4, LDGF, MDGF, NAP-2, |
| | | | SCYB7, TC1, TC2, TGB, TGB1, THBGB, |
| | | | THBGB1, PPBP |
| 1490 | CTGF | 2.10E−06 | CCN2, HCS24, IGFBP8, NOV2, CTGF |
| 22915 | MMRN1 | 2.30E−06 | ECM, EMILIN4, GPIa*, MMRN, MMRN1 |
| 9564 | BCAR1 | 3.30E−06 | CAS, CAS1, CASS1, CRKAS, P130Cas, BCAR1 |
| 3665 | IRF7 | 3.30E−06 | IMD39, IRF-7H, IRF7A, IRF7B, IRF7C, IRF7H, |
| | | | IRF7 |
| 1003 | CDH5 | 3.50E−06 | 7B4, CD144, CDH5 |
| 103 | ADAR | 3.60E−06 | ADAR1, AGS6, DRADA, DSH, DSRAD, G1P1, |
| | | | IFI-4, IFI4, K88DSRBP, P136, ADAR |
| 5340 | PLG | 3.70E−06 | PLG |
| 1675 | CFD | 3.90E−06 | ADIPSIN, ADN, DF, PFD, CFD |
| 10379 | IRF9 | 4.00E−06 | IRF-9, ISGF3, ISGF3G, p48, IRF9 |
| 5552 | SRGN | 4.00E−06 | PRG, PPG, PRG1, SRGN |
| 6041 | RNASEL | 4.10E−06 | RNS4, PRCA1, RNASEL |
| 3339 | HSPG2 | 4.20E−06 | HSPG, PLC, PRCAN, SJA, SJS, SJS1, HSPG2 |
| 710 | SERPING1 | 4.50E−06 | C1IN, C1INH, C1NH, HAE1, HAE2, SERPING1 |
| 5881 | RAC3 | 4.50E−06 | RAC3 |
| 91543 | RSAD2 | 4.70E−06 | 2510004L01Rik, cig33, cig5, vig1, RSAD2 |
| 3684 | ITGAM | 5.00E−06 | CD11B, CR3A, MAC-1, MAC1A, MO1A, SLEB6, |
| | | | ITGAM |
| 3106 | HLA-B | 5.00E−06 | AS, HLAB, SPDA1, HLA-B |
| 6714 | SRC | 5.10E−06 | ASV, SRC1, c-SRC, p60-Src, SRC |
| 81 | ACTN4 | 5.20E−06 | ACTININ-4, FSGS, FSGS1, ACTN4 |
| 7094 | TLN1 | 5.20E−06 | ILWEQ, TLN, TLN1 |
| 3687 | ITGAX | 5.30E−06 | SLEB6, CD11C, ITGAX |
| 87 | ACTN1 | 5.70E−06 | BDPLT15, ACTN1 |
| 3669 | ISG20 | 5.90E−06 | CD25, HEM45, ISG20 |
| 7448 | VTN | 6.10E−06 | V75, VN, VNT, VTN |
| 3481 | IGF2 | 6.20E−06 | C11orf43, GRDF, IGF-II, PP9974, IGF2 |
| 51284 | TLR7 | 6.60E−06 | TLR7-like, TLR7 |
| 6773 | STAT2 | 6.90E−06 | ISGF-3, P113, STAT113, STAT2 |
| 5829 | PXN | 7.00E−06 | PXN |
| 3662 | IRF4 | 7.20E−06 | LSIRF, MUM1, NF-EM5, SHEP8, IRF4 |
| 4322 | MMP13 | 7.80E−06 | CLG3, MANDP1, MMP-13, MMP13 |
| 7114 | TMSB4X | 9.80E−06 | FX, PTMB4, TB4X, TMSB4, TMSB4X |
| 7423 | VEGFB | 9.80E−06 | VEGFL, VRF, VEGFB |
| 7043 | TGFB3 | 1.00E−05 | ARVD, ARVD1, RNHF, TGF-beta3, TGFB3 |
| 6385 | SDC4 | 1.00E−05 | SYND4, SDC4 |
| 3383 | ICAM1 | 1.10E−05 | BB2, CD54, P3.58, ICAM1 |
| 22918 | CD93 | 1.10E−05 | C1QR1, C1qR(P), C1qRP, CDw93, ECSM3, |
| | | | MXRA4, dJ737E23.1, CD93 |
| 2200 | FBN1 | 1.20E−05 | ACMICD, ECTOL1, FBN, GPHYSD2, MASS, |
| | | | MFS1, OCTD, SGS, SSKS, WMS, WMS2, FBN1 |
| 2247 | FGF2 | 1.20E−05 | BFGF, FGF-2, FGFB, HBGF-2, FGF2 |
| 5008 | OSM | 1.30E−05 | OSM |
| 4938 | OAS1 | 1.50E−05 | IFI-4, OIAS, OIASI, OAS1 |
| 3661 | IRF3 | 1.60E−05 | IRF3 |
| 3565 | IL4 | 1.70E−05 | BCGF-1, BCGF1, BSF-1, BSF1, IL-4, IL4 |
| 4317 | MMP8 | 1.90E−05 | CLG1, HNC, MMP-8, PMNL-CL, MMP8 |
| 57180 | ACTR3B | 1.90E−05 | ARP11, ARP3BETA, ACTR3B |
| 5155 | PDGFB | 1.90E−05 | IBGC5, PDGF-2, PDGF2, SIS, SSV, c-sis, PDGFB |
| 7042 | TGFB2 | 1.90E−05 | LDS4, TGF-beta2, TGFB2 |
| 3105 | HLA-A | 1.90E−05 | HLAA, HLA-A |
| 6696 | SPP1 | 2.00E−05 | BNSP, BSPI, ETA-1, OPN, SPP1 |
| 4600 | MX2 | 2.00E−05 | MXB, MX2 |
| 4323 | MMP14 | 2.10E−05 | MMP-14, MMP-X1, MT-MMP, MT-MMP 1, MT1- |
| | | | MMP, MT1MMP, MTMMP1, WNCHRS, MMP14 |
| 4599 | MX1 | 2.20E−05 | IFI-78K, IFI78, MX, MxA, MX1 |
| 5196 | PF4 | 2.30E−05 | CXCL4, PF-4, SCYB4, PF4 |
| 3479 | IGF1 | 2.40E−05 | IGF-I, IGFI, MGF, IGF1 |
| 706 | TSPO | 2.60E−05 | BPBS, BZRP, DBI, IBP, MBR, PBR, PBS, PKBS, |
| | | | PTBR, mDRC, pk18, TSPO |
| 351 | APP | 2.70E−05 | AAA, ABETA, ABPP, AD1, APPI, CTFgamma, |
| | | | CVAP, PN-II, PN2, APP |
| 3660 | IRF2 | 3.00E−05 | IRF-2, IRF2 |
| 3689 | ITGB2 | 3.10E−05 | LAD, LFA-1, MAC-1, CD18, LCAMB, MF17, |
| | | | MFI7, ITGB2 |
| 1311 | COMP | 3.40E−05 | MED, EDM1, EPD1, PSACH, THBS5, COMP |
| 8638 | OASL | 3.50E−05 | OASLd, TRIP-14, TRIP14, p59 OASL, p59-OASL, |
| | | | p59OASL, OASL |
| 5627 | PROS1 | 4.10E−05 | PSA, PROS, PS21, PS22, PS23, PS24, PS25, |
| | | | THPH5, THPH6, PROS1 |
| 3663 | IRF5 | 4.20E−05 | SLEB10, IRF5 |
| 6382 | SDC1 | 4.70E−05 | CD138, SDC, SYND1, syndecan, SDC1 |
| 5653 | KLK6 | 4.70E−05 | Bssp, Klk7, PRSS18, PRSS9, SP59, hK6, KLK6 |
| 3026 | HABP2 | 4.70E−05 | FSAP, HABP, HGFAL, PHBP, HABP2 |
| 4147 | MATN2 | 4.90E−05 | MATN2 |
| 4940 | OAS3 | 5.20E−05 | p100, p100OAS, OAS3 |
| 387 | RHOA | 5.20E−05 | ARH12, ARHA, RHO12, RHOH12, RHOA |
| 10096 | ACTR3 | 5.20E−05 | ARP3, ACTR3 |
| 4921 | DDR2 | 5.30E−05 | MIG20a, NTRKR3, TKT, TYRO10, DDR2 |
| 10561 | IFI44 | 5.50E−05 | p44, MTAP44, TLDC5, IFI44 |
| 88 | ACTN2 | 5.60E−05 | CMD1AA, CMH23, ACTN2 |
| 2 | A2M | 5.80E−05 | A2MD, CPAMD5, FWP007, S863-7, A2M |
| 176 | ACAN | 7.10E−05 | AGC1, AGCAN, CSPG1, CSPGCP, MSK16, |
| | | | SEDK, ACAN |
| 3627 | CXCL10 | 7.10E−05 | C7, IFI10, INP10, IP-10, SCYB10, crg-2, gIP-10, |
| | | | mob-1, CXCL10 |
| 1958 | EGR1 | 7.20E−05 | AT225, G0S30, KROX-24, NGFI-A, TIS8, ZIF-268, |
| | | | ZNF225, EGR1 |
| 3659 | IRF1 | 7.30E−05 | IRF-1, MAR, IRF1 |
| 1191 | CLU | 7.60E−05 | AAG4, APO-J, APOJ, CLI, CLU1, CLU2, KUB1, |
| | | | NA1/NA2, SGP-2, SGP2, SP-40, TRPM-2, TRPM2, |
| | | | CLU |
| 2633 | GBP1 | 8.20E−05 | GBP1 |
| 2153 | F5 | 8.30E−05 | FVL, PCCF, RPRGL1, THPH2, F5 |
| 6387 | CXCL12 | 8.50E−05 | IRH, PBSF, SCYB12, SDF1, TLSF, TPAR1, |
| | | | CXCL12 |
| 1991 | ELANE | 8.60E−05 | ELA2, GE, HLE, HNE, NE, PMN-E, SCN1, |
| | | | ELANE |
| 80162 | ATHL1 | 9.20E−05 | ATHL1 |
| 2534 | FYN | 9.70E−05 | SLK, SYN, p59-FYN, FYN |
| 4939 | OAS2 | 9.80E−05 | OAS2 |
| 325 | APCS | 1.00E−04 | HEL-S-92n, PTX2, SAP, APCS |
| 998 | CDC42 | 1.00E−04 | CDC42Hs, G25K, CDC42 |
| 4035 | LRP1 | 1.10E−04 | LRP, A2MR, APOER, APR, CD91, IGFBP3R, |
| | | | LRP1A, TGFBR5, LRP1 |
| 7124 | TNF | 1.60E−04 | DIF, TNF-alpha, TNFA, TNFSF2, TNF |
| 2162 | F13A1 | 1.90E−04 | F13A, F13A1 |
| 3569 | IL6 | 2.00E−04 | HGF, BSF2, HSF, IFNB2, IL-6, IL6 |
| 3683 | ITGAL | 2.90E−04 | CD11A, LFA-1, LFA1A, ITGAL |
| 11274 | USP18 | 4.20E−04 | ISG43, UBP43, USP18 |
| 3576 | IL8 | 4.30E−04 | GCP-1, GCP1, IL8, LECT, LUCT, LYNAP, |
| | | | MDNCF, MONAP, NAF, NAP-1, NAP1, CXCL8 |
| 1511 | CTSG | 7.00E−04 | CATG, CG, CTSG |
| 4688 | NCF2 | 1.10E−03 | NCF-2, NOXA2, P67-PHOX, P67PHOX, NCF2 |
| 24138 | IFIT5 | 1.80E−03 | P58, ISG58, RI58, IFIT5 |
| 3071 | NCKAP1L | 2.30E−03 | HEM1, NCKAP1L |
| 7097 | TLR2 | 2.60E−03 | CD282, TIL4, TLR2 |
| 9332 | CD163 | 2.70E−03 | M130, MM130, CD163 |
| 6404 | SELPLG | 3.60E−03 | CD162, CLA, PSGL-1, PSGL1, SELPLG |
| 1794 | DOCK2 | 4.20E−03 | IMD40, DOCK2 |
| 8515 | ITGA10 | 5.00E−03 | PRO827, ITGA10 |
| 10437 | IFI30 | 5.20E−03 | GILT, IFI-30, IP-30, IP30, IFI30 |
| 23586 | DDX58 | 5.90E−03 | RIG-I, RIGI, RLR-1, SGMRT2, DDX58 |
| 5217 | PFN2 | 2.00E−02 | D3S1319E, PFL, PFN2 |
| 10346 | TRIM22 | 3.50E−02 | GPSTAF50, RNF94, STAF50, TRIM22 |
| 9938 | ARHGAP25 | 5.60E−02 | HEL-S-308, KAIA0053, ARHGAP25 |
| 716 | C1S | 9.70E−02 | C1S |
| 9672 | SDC3 | 1.10E−01 | SDCN, SYND3, SDC3 |
| 2207 | FCER1G | 1.60E−01 | FCRG, FCER1G |
| 10875 | FGL2 | 1.70E−01 | T49, pT49, FGL2 |
| 6279 | S100A8 | 1.80E−01 | MIF, 60B8AG, CAGA, CFAG, CGLA, CP-10, |
| | | | L1Ag, MA387, MRP8, NIF, P8, S100A8 |
| 2217 | FCGRT | 1.90E−01 | FCRN, alpha-chain, FCGRT |
| 4332 | MNDA | 1.90E−01 | PYHIN3, MNDA |
| 9056 | SLC7A7 | 2.80E−01 | LAT3, LPI, MOP-2, Y + LAT1, y + LAT-1, SLC7A7 |
| 1043 | CD52 | 3.30E−01 | CDW52, CD52 |
| 10261 | IGSF6 | 5.10E−01 | DORA, IGSF6 |

*Gene ID refers to the Entrez Gene identifier

Disorders Associated with Fibrosis

Aspects of the disclosure provide methods for treating disorders associated with fibrosis by administering an agent that modulates expression and/or activity of any of the proteins provided in Table 1. Fibrosis is a condition characterized by excessive production and deposition of extracellular matrix proteins, including collagen and glycosaminoglycans in organs and/or tissues. The process of fibrosis is a normal response to injury or cellular damage, but inappropriate production and accumulation of connective tissue leads to thickening and hardening of the organ and/or tissue, which can result in disruption of normal organ and/or tissue function.

As used herein, a disorder is “associated” with fibrosis if the disorder involves or is characterized by fibrosis. Fibrosis can occur in a variety of tissues or organs. Non-limiting examples of disorders associated with fibrosis include, without limitation, pulmonary fibrosis, cirrhosis, atrial fibrosis, endomyocardial fibrosis, bone marrow fibrosis, glial scar, arthrofibrosis, Crohn's disease, Dupuytren's contracture, keloid, mediastinal fibrosis, myelofibrosis, Peyronie's disease, nephrogenic systemic fibrosis, progressive massive fibrosis, retroperitoneal fibrosis, systemic sclerosis, skeletal muscle fibrosis, and adhesive capsulitis.

Aspects of the present disclosure provide methods of treating a disorder associated with fibrosis by administering an agent to a subject having a disorder associated with fibrosis. In some embodiments, the subject is assessed to determine whether the subject has a disorder associated with fibrosis or to determine the severity of the disorder associated with fibrosis prior to administering the agent. Methods for diagnosing or assessing the severity of disorders associated with fibrosis are known in the art.

### Systemic Sclerosis

Aspects of the disclosure provide methods for treating systemic sclerosis by administering an agent that modulates expression and/or activity of any of the proteins provided in Table 1. “Systemic sclerosis” is also referred to as “scleroderma” or “progressive systemic sclerosis” and is a chronic autoimmune disorder with unknown etiology. Systemic sclerosis is generally characterized by vascular damage, immune activation, and excessive production of extracellular matrix proteins including collagen, fibronectin, tenascin, fibrillin, and glycosaminogclyans. Although systemic sclerosis remains poorly understood, it is generally considered that vascular injury due to an autoimmune response may trigger constitutive activation of fibroblasts and fibrosis (Del Papa et al. Best Pract. Res. Clin. Rheumatol. 29(6): 756-9). Accumulation of the extracellular matrix proteins from fibroblasts leads to thickening and hardening of the skin or tissue, but can also include additional organs, eventually resulting in organ dysfunction and failure. When limited to the skin, systemic sclerosis frequently causes fibrosis of the skin of the face, neck, elbows, and knees, and in such cases is referred to as limited cutaneous systemic sclerosis. The disorder may also progress to involve more of the skin as well as visceral organs, such as the heart, lungs, kidneys, and gastrointestinal tract, which is referred to as diffuse cutaneous systemic sclerosis (Steen et al. Arthritis Rheum. 43(11): 2437-44). The severity and prognosis of diffuse cutaneous systemic sclerosis is determined by assessing the extent of visceral organ involvement (Hinchcliff et al. Am. Fam. Physician. (2008) 78(8): 961-8). Pulmonary fibrosis, pulmonary hypertension, severe gastrointestinal disorders, and scleroderma heart disease are the primary causes of death related to systemic sclerosis.

Due to the absence of any effective disease-modifying therapies, current treatment regimens aim to reduce the symptoms of systemic sclerosis and slow disease progression, for example by improving vascular circulation, promoting gastrointestinal function, controlling hypertension, promoting kidney function, and generally preventing serious complications.

In addition to thickening and hardening of the skin, additional symptoms of systemic sclerosis may include, for example, joint pain, reduced joint motility, muscle weakness or pain, Raynaud's phenomenon, swelling of the fingers or toes, ulcers present on the fingertips, fatigue, weight loss, heartburn, bloating, constipation, scarring in the lungs, and hypertension.

Aspects of the present disclosure provide methods of treating systemic sclerosis by administering an agent to a subject having systemic sclerosis. In some embodiments, the subject is assessed to determine whether the subject has systemic sclerosis or to determine the severity of the systemic sclerosis prior to administering the agent. Methods for diagnosing or assessing the severity of systemic sclerosis are known in the art, and may include, for example, clinical presentation; modified Rodnan skin score; presence of autoantibodies, such as anti-nuclear antibodies (e.g., anti-centromere antibodies, anti-topoisomerase antibodies, anti-RNA polymerase III antibodies); and/or expression of one or more biomarkers associated with systemic sclerosis (e.g., TGF-β, TGF-β-regulated genes, COMP, CTGF, PAIL THS1, COL4, IFN-regulated genes, IFI-44, OAS2, Sig-1, and MA-1). See, e.g., PCT Application Nos. WO 2013/149927 and WO 2012/140209, and U.S. Publication 2011/0189682, herein incorporated by reference in their entireties.

### Agents

Aspects of the disclosure relate to the use of agents that modulate expression and/or activity of any of the proteins provided in Table 1. As described herein, the term “modulate” encompasses inhibiting (decreasing) and enhancing (increasing) expression and/or activity of a protein. An agent may modulate the expression and/or activity of a protein by any mechanism known in the art. In some embodiments, the agent selectively modulates the expression and/or activity of a protein provided in Table 1. As used herein, an agent that “selectively modulates” a protein refers to an agent that preferentially modulates one or a small number of related proteins. In some embodiments, an agent modulates a group or class of proteins.

In some embodiments, the agent modulates expression of a protein provided in Table 1, for example by modulating the expression of a nucleic acid encoding any of the proteins in Table 1. In general, expression of a nucleic acid encoding a protein can be modulated by any of a variety of methods, for example by modulating transcription, mRNA localization, mRNA degradation, mRNA stability, and/or translation of the protein. In some embodiments, the agent modulates expression of a nucleic acid by promoting or inhibiting transcription of the nucleic acid. In other embodiments, the agent modulates expression of a nucleic acid by promoting or inhibiting mRNA localization, mRNA degradation or mRNA stability. In other embodiments, the agent modulates expression of a nucleic acid by promoting or inhibiting translation of the nucleic acid. In other embodiments, an agent modulates protein levels by modulating protein stability or protein degradation.

In some embodiments, the agent inhibits expression of the protein, such that the amount of the protein or the amount of a nucleic acid encoding the protein is reduced relative to the amount of the protein or the amount of the nucleic acid encoding the protein in the absence of the agent. In some embodiments, the amount of the protein or the amount of a nucleic acid encoding the protein is reduced by at least 1.5-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 500-, or at least 1000-fold or more relative to the amount of the protein or the amount of the nucleic acid encoding the protein in the absence of the agent. In some embodiments, the amount of the protein or the amount of a nucleic acid encoding the protein in the presence of the agent is about 5%, 10%, 15%, 20%, 25%, 30%, 35%, 40%, 45%, 50%, 55%, 60%, 65%, 70%, 75%, 80%, 85%, 90%, or about 95% of the amount of protein or nucleic acid encoding the protein that is produced in the absence of the agent. In some embodiments, the protein is not detectably expressed, i.e., there is no detectable protein and/or nucleic acid encoding the protein, following administration of the agent.

In some embodiments, the agent enhances expression of a protein in Table 1, such that the amount of the protein or the amount of a nucleic acid encoding the protein is enhanced relative to the amount of the protein or the amount of the nucleic acid encoding the protein in the absence of the agent. In some embodiments, the amount of the protein or the amount of a nucleic acid encoding the protein is enhanced by at least 1.5-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 500-, or at least 1000-fold or more relative to the amount of the protein or the amount of the nucleic acid encoding the protein in the absence of the agent. In some embodiments, the amount of the protein or the amount of a nucleic acid encoding the protein in the presence of the agent is about 5%, 10%, 15%, 20%, 25%, 30%, 35%, 40%, 45%, 50%, 55%, 60%, 65%, 70%, 75%, 80%, 85%, 90%, or about 95% more than the amount of protein or nucleic acid encoding the protein that is produced in the absence of the agent.

The agent can modulate the activity of a protein provided in Table 1 with or without modulation of the nucleic acid encoding the protein. In some embodiments, the agent interacts with the protein directly or indirectly, thereby affecting the activity of the protein. In some embodiments, the agent may modulate the activity of a protein by modulating protein stability, protein degradation, one or more protein interactions, enzymatic activity, conformation, and or signaling activity. In some embodiments, an agent eliminates the activity of a protein. In other embodiments, an agent renders a protein constitutively active.

In some embodiments, the agent inhibits activity of a protein in Table 1, such that the activity of the protein is reduced relative to the activity of the protein in the absence of the agent. In some embodiments, the activity of the protein is reduced by at least 1.5-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 500-, or at least 1000-fold or more relative to the activity of the protein in the absence of the agent. In some embodiments, there is no detectable protein activity following administration of the agent, i.e., activity of the protein is completely inhibited. In some embodiments, the activity of the protein in the presence of the agent is about 5%, 10%, 15%, 20%, 25%, 30%, 35%, 40%, 45%, 50%, 55%, 60%, 65%, 70%, 75%, 80%, 85%, 90%, or about 95% of the activity of the protein in the absence of the agent.

In some embodiments, the agent enhances activity of the protein, such that the activity of the protein is enhanced relative to the activity of the protein in the absence of the agent. In some embodiments, the activity of the protein is enhanced by at least 1.5-, 2-, 3-, 4-, 5-, 6-, 7-, 8-, 9-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-, 500-, or at least 1000-fold or more relative to the activity of the protein in the absence of the agent. In some embodiments, the activity of the protein in the presence of the agent is about 105%, 110%, 115%, 120%, 125%, 130%, 140%, 150%, 160%, 170%, 180%, 190%, 200%, 225%, 250%, 275%, 300%, 325%, 350%, 375%, 400%, 425%, 450%, 475%, 500%, 550%, 600%, 650%, 700%, 750%, 800%, 850%, 900%, 950%, or about 1000% or more of the activity of the protein in the absence of the agent.

Methods for assessing the expression and/or activity of a protein will be evident to one of ordinary skill in the art and can be conducted in vitro or in vivo. Methods may involve collecting one or more biological sample from a subject In some embodiments, expression and/or activity of the protein is assessed prior to and/or after administration of the agent to the subject. Methods can involve measuring the level of mRNA and/or protein, and/or measuring the activity of a protein, such as the enzymatic activity or the signaling activity of a protein.

An agent that modulates the expression and/or activity of a protein may be in any form known in the art. For example, in some embodiments, the agent is an antibody or fragment thereof, a protein, a fusion protein, a small molecule, or a nucleic acid.

In some embodiments, the agent is an antibody (or portion thereof) that modulates the expression and/or activity of a protein presented in Table 1. As used herein, the term “antibody” refers to an immunoglobulin molecule, including functional fragments thereof, that binds to an epitope of an antigen (e.g., a protein provided in Table 1). In some embodiments, the antibody selectively binds to an epitope of a protein provided in Table 1. As used herein, “selectively binds” means that an antibody preferentially binds to an epitope of a protein provided in Table 1 (e.g., with greater avidity, greater binding affinity) rather than to another protein. In some embodiments, the antibody is a naturally occurring antibody (e.g., an antibody from a suitable source, such as a human, mouse, rat, rabbit, horse, goat, or sheep), derived from a naturally occurring antibody, an engineered antibody (e.g., a fully human antibody, a humanized antibody, or a chimeric antibody), or derived from a synthetic antibody. The antibodies described herein may be in any form, such as full-length antibodies, or antigen-binding fragments thereof, such as Fab, Fab′, F(ab′)₂, Fv), single chain antibodies (scFv), dsFv, scdsFv, diabody, or single-domain antibodies (nanobodies). The antibody may be of any isotype, such as IgG, IgA, IgM, IgE, or IgE, or any subclass thereof.

In some embodiments, the antibody inhibits expression and/or activity of a protein presented in Table 1. In some embodiments, the antibody enhances expression and/or activity of a protein presented in Table 1. In some embodiments, the antibody modulates expression of the protein by inhibiting or preventing transcription of a nucleic acid encoding the protein, for example by interacting with one or more components involved in the transcription process. In some embodiments, the antibody modulates expression of the protein by inhibiting or preventing translation of the protein, for example by interacting with one or more of the components involved in the translation process. In some embodiments, the antibody modulates activity of the protein, for example by interacting with the protein directly or indirectly. In some embodiments, the antibody modulates the activity of the protein by modulating protein stability, protein degradation, one or more protein interactions, enzymatic activity, conformation, and or signaling activity. In some embodiments, an antibody eliminates the activity of a protein. In other embodiments, an antibody renders a protein constitutively active.

In some embodiments, the agent is a protein or fusion protein that modulates the expression and/or activity of a protein presented in Table 1. In some embodiments, the protein is a recombinant protein. As used herein, a “fusion protein” refers to a protein comprised of one or more proteins or portions thereof. For example, a portion of a first protein may be combined with a portion of a second protein to form a fusion protein. In some embodiments, the fusion protein is an Fc fusion protein, in which the Fc domain of an antibody is combined with a portion of another protein. In some embodiments, the protein or fusion protein inhibits expression and/or activity of a protein presented in Table 1. In some embodiments, the protein or fusion protein enhances expression and/or activity of a protein presented in Table 1. In some embodiments, the protein or fusion protein modulates expression of the protein by inhibiting or preventing transcription of a nucleic acid encoding the protein, for example by interacting with one or more components involved in the transcription process. In some embodiments, the protein or fusion protein modulates expression of the protein by inhibiting or preventing translation of the protein, for example by interacting with one or more of the components involved in the translation process. In some embodiments, the protein or fusion protein modulates activity of a protein, for example by interacting with the protein directly or indirectly.

In some embodiments, the agent is a small molecule that modulates the expression and/or activity of a protein presented in Table 1. As used herein, a “small molecule,” including small molecule inhibitors and small molecule activators, refers to a compound having a low molecular weight (e.g., less than 900 Daltons). In some embodiments, the small molecule inhibits expression and/or activity of a protein presented in Table 1. In some embodiments, the small molecule enhances expression and/or activity of a protein presented in Table 1. In some embodiments, the small molecule modulates expression of the protein by inhibiting or preventing transcription of a nucleic acid encoding the protein, for example by interacting with one or more components involved in the transcription process. In some embodiments, the small molecule modulates expression of the protein by inhibiting or preventing translation of the protein, for example by interacting with one or more of the components involved in the translation process. In some embodiments, the small molecule modulates activity of a protein, for example by interacting with the protein directly or indirectly.

In some embodiments, the agent is a nucleic acid that modulates the expression and/or activity of a protein presented in Table 1. In some embodiments, the nucleic acid inhibits expression and/or activity of a protein presented in Table 1. In some embodiments, the nucleic acid enhances expression and/or activity of a protein presented in Table 1. In some embodiments, the nucleic acid modulates expression of the protein by inhibiting or preventing transcription of a nucleic acid encoding the protein. In some embodiments, the nucleic acid modulates expression of the protein by inhibiting or preventing translation of the protein and/or by modulating mRNA degradation. In some embodiments, the nucleic acid modulates the activity of the protein, for example through protein-nucleic acid interactions. Examples of nucleic acids that may modulate the expression and/or activity of a protein presented in Table 1 include, without limitation, double-stranded RNA molecules, single-stranded RNA molecules, antisense oligonucleotides, microRNAs (miRNAs), shRNAs, siRNAs, and CRISPR/Cas guide RNAs.

In some embodiments, the agent that modulates the expression and/or activity of one or more proteins provided in Table 1 is selected from any of the example agents provided in Table 3.

TABLE 3
Examples of Agents Targeting Proteins in Table 1

| Gene ID* | Symbol | Known Therapeutics |
|---|---|---|
| 3676 | ITGA4 | Natalizumab is a humanized mAb that blocks the passage of |
| | | leucocytes across the blood/brain barrier, developed by |
| | | Perrigo (Elan before acquisition) and Biogen Idec for the |
| | | treatment of multiple sclerosis (MS), Crohn's disease (CD) |
| | | and other inflammatory disorders. It was also in development |
| | | for multiple myeloma. It is directed against VLA-4. Biogen |
| | | Idec subsequently acquired all rights. |
| | | Vedolizumab is an injectable humanized monoclonal |
| | | antibody to alpha4ß7 integrin, developed by Millennium (now |
| | | Takeda) (LeukoSite before acquisition) for the treatment of |
| | | Crohn's disease (CD), ulcerative colitis (UC) and irritable |
| | | bowel syndrome (IBS). ß7 integrin is found on T-lymphocyte |
| | | memory cells and modulates their binding to MAdCAM-1 (an |
| | | adhesion receptor on the cells of blood vessels lining the |
| | | intestine). Millennium was also independently developing |
| | | small-molecule ß7 integrin antagonists for chronic use. |
| 3678 | ITGA5 | Phase II. ALG-1001 (Luminate) is an anti-integrin |
| | | oligopeptide, under development by Allegro Ophthalmics for |
| | | the treatment of diabetic macular oedema (DMO), wet AMD |
| | | and symptomatic focal vitreomacular adhesion |
| 3680 | ITGA9 | GND-001 is an anti-alpha9 integrin monoclonal antibody |
| | | (mAb), under development by Gene Techno Science (GTS) |
| | | for the treatment of autoimmune diseases and cancer |
| | | metastasis (Press release, Kaken, 4 Jul. 2007; Annual Report, |
| | | Kaken, 2010, Page 12, |
| | | www.kaken.co.jp/english/ar/ar/ar_2010.pdf; Company |
| | | pipeline, GTS, 16 Jan. 2015, ir.g- |
| | | gts.com/ja/vision/pipeline.html). |
| 3688 | ITGB1 | Natalizumab is a humanized mAb that blocks the passage of |
| | | leucocytes across the blood/brain barrier, developed by |
| | | Perrigo (Elan before acquisition) and Biogen Idec for the |
| | | treatment of multiple sclerosis (MS), Crohn's disease (CD) |
| | | and other inflammatory disorders. It was also in development |
| | | for multiple myeloma (Scrip, 1991, 1661, 11; Company Web |
| | | Page, Elan, 24 Feb. 2004; Press release, Perrigo, 18 Dec. 2013, |
| | | perrigo.investorroom.com/2013-12-18-Perrigo-Company-plc- |
| | | Completes-Acquisition-of-Elan-Corporation-plc). It is |
| | | directed against VLA-4 (Scrip, 1994, 1977, 27). Biogen Idec |
| | | subsequently acquired all rights (Press release, Biogen, 2 Apr. |
| | | 2013, |
| | | www.biogenidec.com/press_release_details.aspx?ID=5981& |
| | | ReqId=1802638). |
| 22801 | ITGA11 | Hansa Medical has reportedly discontinued development of |
| | | an integrin alpha11ß1 antagonist for the treatment of |
| | | osteoarthritis and rheumatoid arthritis. Hansa Medical will, |
| | | however, reportedly continue conducting research on alpha- |
| | | 11 as a drug target for diseases other than rheumatoid arthritis |
| | | (Company Web Page, Hansa, 29 Jul. 2008; BIO 2010 |
| | | (Chicago), www.bio.org/bfprofiles/pdfs/84877.pdf; Press |
| | | release, Hansa, 9 Feb. 2012, |
| | | feed.ne.cision.com/wpyfs/00/00/00/00/00/18/34/FB/release.html). |
| 3693 | ITGB5 | Phase II |
| 5747 | PTK2 | Phase II |
| 960 | CD44 | ONCOFID ™-S, bioconjugate of hyaluronic acid (HA) with |
| | | SN-38 (the CPT11 active metabolite) |
| 4313 | MMP2 | PHY-906 is a broad spectrum botanical extract, under co- |
| | | development by Kadmon Pharmaceuticals (PhytoCeutica |
| | | before acquisition) and Lumosa Therapeutics (SunTen |
| | | Phytotech before merger) for the treatment of severe diarrhea |
| | | associated with chemotherapy for colorectal cancer, Crohn's |
| | | disease and hepatocellular carcinoma. It targets the inhibition |
| | | of MP-2, MDR, NFkB, beta-glucuronidase, NK-1 receptor |
| | | and the delta opioid receptor and is designed to confer GI |
| | | protection (anti-diarrheal, nausea and vomiting, cramps and |
| | | distension), antipyretic, antioxidant, antiviral (immunoligic |
| | | protection), analgesic and hepatoprotection (Company Web |
| | | Page, PhytoCeutica, 26 Feb. 2003 & SunTen, 26 Mar. 2007; |
| | | Company pipeline, Kadmon, 16 Sep. 2011, |
| | | kadmon.com/docs/science_pipeline). It is a standardized 4- |
| | | herb traditional Chinese formulation that has been used for |
| | | over 1700 yr in the treatment of gastrointestinal ailments |
| | | (100th AACR (Denver), 2009, Abs 4584). |
| 7450 | VWF | Launched |
| 3655 | ITGA6 | Dyax (Shire) was reportedly developing therapeutics targeting |
| | | a truncated form of the alpha-6 integrin receptor associated |
| | | with precancerous prostate lesions and invasive prostate |
| | | cancer, using phage display technology to identify human |
| | | antibodies and peptides that bind to the receptor. |
| 3674 | ITGA2B | Abciximab is a monoclonal antibody licensed by Centocor |
| | | (Johnson & Johnson; J&J) from the State University, New |
| | | York, US, for the treatment of clot-related cardiovascular |
| | | disease. It blocks the GPIIb/IIIa fibrinogen binding site on |
| | | platelets to prevent aggregation, and the vitronectin receptor |
| | | and inhibits the ß2-integrin, MAC-1 (Scrip, 1997, 2259, 17; |
| | | Press release, Centocor & Lilly, 4 May 2000). |
| 3695 | ITGB7 | Many therapeutics targeting this agent are in development or |
| | | launched for different AIs |
| 1634 | DCN | Telios (Integra LifeSciences) has reportedly discontinued |
| | | development of a recombinant form of human decorin, a |
| | | protein with strong inhibitory activity against TGF-ß1, as an |
| | | antifibrotic agent |
| 3685 | ITGAV | Many therapeutics, including some in lung fibrosis |
| 3673 | ITGA2 | Vatelizumab (SAR-339658 (formerly CHR-1103)) is a |
| | | humanized mAb targeting alpha2ß1 integrin (VLA2), under |
| | | development by Glenmark for the treatment of acute relapse |
| | | in multiple sclerosis (MS). It was under development for |
| | | Crohn's disease and ulcerative colitis (Press release, Chromos, |
| | | 15 Aug. 2006; BIO 2009 (Atlanta); Company Pipeline, Sanofi, |
| | | 6 Feb. 2014). It is designed to reduce and prevent the |
| | | accumulation of inflammatory cells within inflamed tissues |
| | | (Press release, Chromos, 25 Jan. 2007). It also had potential in |
| | | oncology (Company presentation, Glenmark, November 2008). |
| 2335 | FN1 | Phase III |
| 4312 | MMP1 | Launched |
| 4318 | MMP9 | Phase III |
| 3675 | ITGA3 | CytomX Therapeutics is developing compounds targeting |
| | | CD71 & ITGA3, using its probody drug conjugate (PDC) |
| | | platform for the treatment of cancer (Company Web Page, |
| | | CytomX, 26 Oct. 2015, cytomx.com/cyt01/pipeline-02/). |
| 7040 | TGFB1 | Many therapeutics |
| 7412 | VCAM1 | Discontinued, AR |
| 3107 | HLA-C | Phase III |
| 2277 | FIGF | Phase III |
| 3273 | HRG | Phase III |
| 7057 | THBS1 | Preclinical |
| 284 | ANGPT1 | Trebananib (AMG-386) is a recombinant Fc fusion protein |
| | | containing an active peptide (peptibody) targeting |
| | | angiopoietin 1 and 2, thereby inhibiting Tie-2-dependent |
| | | stimulation of endothelial cells, under development by |
| | | Amgen for the treatment of cancer |
| 7098 | TLR3 | Phase II |
| 1490 | CTGF | Phase II |
| 1003 | CDH5 | Phase III |
| 5340 | PLG | Launched |
| 1675 | CFD | Phase III |
| 3339 | HSPG2 | RUS-3108 is an oral perlecan inducer, which was under |
| | | development by Dr Reddy's (Perlecan Pharma before merger) |
| | | for the treatment of atherosclerosis |
| 710 | SERPING1 | Launched. Conestat alfa (Rhucin; rhC1INH) is a recombinant |
| | | human C1 esterase inhibitor produced in the milk of |
| | | transgenic rabbits, developed by Pharming for the treatment |
| | | of angioedema. |
| 3684 | ITGAM | anti-CD11b monoclonals, Repl; RepliGen was developing |
| | | several monoclonal antibodies (mAbs) as anti-inflammatory |
| | | agents. |
| 3106 | HLA-B | Vical discontinued development of velimogene aliplasmid |
| | | (Allovectin-7; Allovectin), a gene therapy product, for the |
| | | treatment of cancer, after a Phase III trial failed to meet its |
| | | endpoints (USAN Web Page, 2 Jul. 2008). It was a complex of |
| | | the MHC class I foreign tissue antigen HLA-B7 and ß2 |
| | | microglobulin genes with a cationic lipid (cytofectin) which |
| | | permitted cellular transfection. |
| 6714 | SRC | Many cancer therapeutics |
| 3687 | ITGAX | Discontinued. Glaxo Wellcome (now GlaxoSmithKline) was |
| | | developing binding agents specific to CD23, CD11b, CD11c |
| | | and CD21 for use in the treatment of inflammatory, |
| | | autoimmune and allergic diseases (WO9612741 & |
| | | WO9612742). |
| 7448 | VTN | Crucell (Johnson & Johnson; formerly U-BiSys) and the |
| | | University Medical Center, Utrecht, the Netherlands, were |
| | | collaborating on the development of an anti-Vn fully-human |
| | | monoclonal antibody as an angiogenesis inhibitor for the |
| | | treatment of solid tumours ((BIO 2001 (San Diego); |
| | | Company Web Page, Crucell, 16 Oct. 2003). Crucell was also |
| | | separately developing another antiangiogenic antibody, |
| | | FibMAb. |
| 3481 | IGF2 | GTI-4006 is an antisense therapeutic that targets insulin-like |
| | | growth factor-2, which was under development by Lorus |
| | | Therapeutics for the treatment of cancer (Ann Rep, Lorus, |
| | | 2002). |
| 51284 | TLR7 | Phase II. DV-1179 is an oligonucleotide-based bifunctional |
| | | TLR 7 and 9 inhibitor, under development by Dynavax for the |
| | | treatment of immuno-inflammatory diseases such as psoriasis, |
| | | cutaneous lupus and related skin conditions, and |
| | | dermatomyositis. |
| 4322 | MMP13 | Searle (Monsanto; now Pfizer) has discontinued the |
| | | development of SD-2590, a matrix metalloproteinase 2, 9 and |
| | | 13 inhibitor, as an anticancer agent |
| 7114 | TMSB4X | Phase III. RegeneRx Biopharmaceuticals (previously Alpha 1 |
| | | Biomedicals) is developing a synthetic version of the 43- |
| | | amino-acid peptide thymosin ß4 (TB4), an MMP-9 and |
| | | MMP-1 modulator that was originally isolated from the |
| | | thymus, as a wound-healing agent for the treatment of dermal |
| | | (RGN-137; topical gel), ophthalmic (RGN-259) and |
| | | cardiovascular indications (RGN-352; injectable formulation) |
| | | as well as cystic fibrosis (CF) and bronchiectasis (RGN-457; |
| | | inhalable formulation) |
| 7423 | VEGFB | CSL346 (2H10) is a humanized mAb that antagonizes |
| | | vascular endothelial growth factor B (VEGF-B), under |
| | | development by CSL for the treatment of Type 2 diabetes and |
| | | diabetes complications |
| 7043 | TGFB3 | Phase II. Fresolumimab (GC-1008; GZ-402669) is a pan- |
| | | specific IgG4 anti-TGF-ß human MAb, under joint- |
| | | development by Cambridge Antibody Technology (CAT) |
| | | (AstraZeneca) and Genzyme (Sanofi) for the treatment of |
| | | fibrotic disease (including renal sclerosis) and melanoma |
| 3383 | ICAM1 | Alicaforsen (ISIS-2302) is an ICAM-1 phosphorothioate |
| | | antisense oligonucleotide, targeted at the 3′-untranslated |
| | | region of ICAM-1, under development by Ionis |
| | | Pharmaceuticals (formerly Isis Pharmaceuticals) for the |
| | | treatment of ulcerative colitis (UC) and other bowel disorders |
| 2247 | FGF2 | Many therapeutics; targeted in pulmonary fibrosis |
| 5008 | OSM | GSK-2330811 is a humanized monoclonal antibody that |
| | | blocks Oncostatin M (OSM), under development by |
| | | GlaxoSmithKline for the treatment of fibrotic diseases and |
| | | scleroderma (ClinicalTrials.gov, 12 Mar. 2015, |
| | | clinicaltrials.gov/ct2/show/NCT02386436). |
| 3565 | IL4 | SAR-156597 is a bi-specific IL4/IL13 antibody, under |
| | | development by Sanofi for the treatment of idiopathic |
| | | pulmonary fibrosis (IPF) |
| 4317 | MMP8 | Glucosamine sulfate has been developed by Rottapharm |
| | | Madaus (now Meda (Mylan)) for use in osteoarthritis (OA) |
| 5155 | PDGFB | Many therapeutics; some in SSc |
| 7042 | TGFB2 | Trabedersen (AP-12009) is an antisense therapy, targeted to |
| | | transforming growth factor (TGF)-ß2, under development by |
| | | Autotelic for the treatment of cancer. It was previously under |
| | | development by Isarna Therapeutics (formerly Antisense |
| | | Pharma) |
| 3105 | HLA-A | ITK-1 is a personalized peptide vaccine against the HLA-A24 |
| | | antigen, under development by GreenPeptide for the |
| | | treatment of cancer |
| 6696 | SPP1 | Brefelamide is a novel inhibitor of osteopontin, under |
| | | development by Fuso Pharmaceuticals for the treament of |
| | | cancer |
| 4323 | MMP14 | 3-D Pharmaceuticals (now Johnson & Johnson) has |
| | | reportedly discontinued development of inhibitors of |
| | | membrane-type 1 matrix metalloproteinase (MT1-MMP) |
| | | inhibitors for the treatment of tumour angiogenesis, restenosis |
| | | and atherosclerosis |
| 3479 | IGF1 | BVS-857 is a version of IGF-1, under development by |
| | | Novartis for the treatment of insulin resistance |
| | | (ClinicalTrials.gov, 5 Oct. 2011 & 11 Oct. 2012, |
| | | clinicaltrials.gov/show/NCT01435330). It is also under |
| | | development for spinal and bulbar muscular atrophy (SBMA) |
| 706 | TSPO | ONO-2952 is an antagonist of translocator protein 18 kDa |
| | | (also called the peripheral benzodiazepine receptor (PBR)), |
| | | under development by Ono for the treatment of IBS |
| 351 | APP | Gantenerumab (R-1450) is a human mAb that targets |
| | | abnormal build-up of amyloid-ß protein in cerebral tissue, |
| | | under development by MorphoSys and Roche for the |
| | | treatment of Alzheimer's disease (AD) |
| 3689 | ITGB2 | Abciximab is a monoclonal antibody licensed by Centocor |
| | | (Johnson & Johnson; J&J) from the State University, New |
| | | York, US, for the treatment of clot-related cardiovascular |
| | | disease. It blocks the GPIIb/IIIa fibrinogen binding site on |
| | | platelets to prevent aggregation, and the vitronectin receptor |
| | | and inhibits the ß2-integrin, MAC-1 |
| 5627 | PROS1 | Integrated Genetics (Genzyme (now Sanofi)) cloned and |
| | | expressed the gene for human protein S. Protein S is a |
| | | cofactor to the anticoagulant protein C; together they break |
| | | down Factors Va and VIIIa. |
| 6382 | SDC1 | Indatuximab ravtansine (BT-062) is a conjugate of a Biotest |
| | | chimaeric mAb targeting CD138 and ImmunoGen's cell- |
| | | killing agent maytansinoid derivates (DM1 and DM4), under |
| | | development by Biotest for the treatment of multiple |
| | | myeloma (MM) and other cancers |
| 5653 | KLK6 | Sinobiomed (Shanghai Wanxing before acquisition) was |
| | | developing a recombinant human type 1 kallikrein (rhK1), for |
| | | the treatment of peripheral vascular disorders, prevention of |
| | | blood clots and thrombosis |
| 387 | RHOA | NOVO-117 is a Rho kinase inhibitor, under development by |
| | | Novoron Bioscience for the treatment of multiple sclerosis. |
| | | NOVO-117 acts on a previously unknown mediator of RhoA |
| | | activation in CNS disease, LRP1 (low density lipoprotein- |
| | | related protein 1) |
| 2 | A2M | Otsuka has reportedly discontinued development of API- |
| | | 13782, an alpha-2-macroglobulin inhibitor as a thrombolytic |
| 176 | ACAN | Phase II. SB-061 is an ECM-specific compound based on |
| | | aggrecans, under development by Symic Biomedical for the |
| | | treatment of osteoarthritis. It mimics the protective |
| | | proteoglycan aggrecan, to reduce cartilage degradation and |
| | | pain |
| 3627 | CXCL10 | Bristol-Myers Squibb is developing interferon-inducible |
| | | protein 10 (IP-10) antagonists for the treatment of |
| | | immunological diseases |
| 1958 | EGR1 | GlaxoSmithKline was developing early growth factor 1 (Erg- |
| | | 1) gene therapy, for use in the acceleration of wound healing. |
| | | Erg-1 is a cellular transcription factor that is naturally |
| | | expressed minutes after acute injury and whose role is to |
| | | stimulate production of growth factors involved in tissue |
| | | repair. |
| 1191 | CLU | Phase I. AB-16B5 is the lead humanized IgG2 mAb in a |
| | | series of clusterin inhibitors, under development by Alethia |
| | | Biotherapeutics for the treatment of breast, colon, lung and |
| | | pancreatic cancer |
| 2633 | GBP1 | Austrianova (now Nuvilex (SGaustria before acquisition)) |
| | | was developing a gene therapy for the treatment of cancer, |
| | | which delivers GBP1, a regulator of angiogenesis |
| 2153 | F5 | Crucell (now Johnson & Johnson) has discontinued |
| | | development of a Factor V molecule, expressed in its |
| | | proprietary PER.C6 cell line, for treatment of haemophilia. |
| 6387 | CXCL12 | Olaptesed pegol (NOX-A12) is an aptamer against chemokine |
| | | SDF1, under development by Noxxon using its Spiegelmer |
| | | technology, for the treatment of haematological and solid |
| | | tumours, and for use in stem cell mobilization. It is a 45- |
| | | nucleotide L-RNA oligonucleotide linked to 40 kDa PEG. It is |
| | | intended for IV and sc administration. It has a half-life of |
| | | approximately 37 hr. |
| 1991 | ELANE | Zemaira (CR-002) is a highly purified sterile, stable, |
| | | lyophilized preparation of highly purified human alpha-1 |
| | | protease inhibitor (A1-PI-IV), developed by Aventis Behring |
| | | (now CSL Behring; formerly ZLB Behring (CSL)) for the |
| | | treatment of congenital emphysema caused by alpha-1 |
| | | antitrypsin (AAT) deficiency (Company Document, CSL, |
| | | November 2011, www.cslbehring-us.com/docs/25/148/Zemaira- |
| | | Prescribing-Information,0.pdf; Company Presentation, CSL, 6 |
| | | Dec. 2012, www.csl.com.au/docs/568/221/RnD_Presentation- |
| | | FINAL-DECEMBER-2012.pdf). The system was to |
| | | incorporate Nektar Therapeutic's non-invasive pulmonary |
| | | drug delivery system, Inhance, with ZLB Behring's alpha-1 |
| | | protease inhibitor. |
| 2534 | FYN | Masitinib mesylate (AB-1010) is a PDGF receptor tyrosine |
| | | kinase, FGFR3, c-kit, Lyn and Fyn tyrosine kinases inhibitor, |
| | | under development by AB Science for the treatment of |
| | | Alzheimer's disease, primary and secondary progressive |
| | | multiple sclerosis (PPMS, SPMS), major depression and |
| | | several cancers, including GIST, rheumatoid arthritis (RA), |
| | | asthma and multiple myeloma (MM) |
| 325 | APCS | PRM-151 is a recombinant human serum amyloid P (rhSAP; |
| | | Pentraxin-2; PTX-2), under development by Promedior for |
| | | the treatment of fibrotic disorders and inflammatory |
| | | conditions of the eye, lung and kidney (BIO 2009 (Atlanta); |
| | | Press releases, Promedior, 23 Jul. 2009 & 16 Mar. 2010; Press |
| | | release, Promedior, 7 Sep. 2010, |
| | | www.promedior.com/news/releases/2010%200907%20Ph2% |
| | | 20Glaucoma.html; Press release, Promedior, 2 Nov. 2011, |
| | | www.promedior.com/news/releases/2011%201102.html). |
| 8515 | ITGA10 | n-CoDeR osteoarthritis are antibody-based inhibitors of |
| | | integrins alpha10ß1 and alpha11ß1 |
| 7124 | TNF | Adalimumab (Humira ®) is an anti-TNF mAb |
| 9332 | CD163 | TBI-304 (formerly HRC-304) is a mAb against CD163 |
| 3694 | ITGB6 | STX-100 is a humanized mAb targeting the alphavß6 |
| | | integrin; Abituzumab is an anti-angiogenic humanized mAb |
| | | (inhibitor of integrin-alpha V ß1, 3, 5 and 6) |
| 10875 | FGL2 | A mAb against fibrinogen-Like 2 (Fgl2) |
| 3683 | ITGAL | Efalizumab is a humanized anti-CD11a mAb |
| 1511 | CTSG | Many inhibitors have been developed targeting this protein |
| 1043 | CD52 | GZ-402668 (GLD-52) is an anti-CD52 humanized Mab |
| 6404 | SELPLG | Neihulizumab (AbGn-168) is a humanized MAb against |
| | | CD162 |
| 7097 | TLR2 | OPN-305 is a fully humanized IgG4 mAb against toll-like |
| | | receptor 2 |
| 3569 | IL6 | Tocilizumab is a humanized mAb against IL-6R |
| 2217 | FCGRT | M281 is an anti-FcRn antibody |
| 3576 | IL8 | Many therapeutics have been developed targeting this protein |
| 6279 | S100A8 | Paquinimod (ABR-215757) is an immunomodulatory small |
| | | quinoline molecule that blocks S100A9 interactions with |
| | | RAGE/TLR4 |

*Gene ID refers to the Entrez Gene identifier

### Subject

In one aspect, the disclosure provides methods of treating a disorder associated with fibrosis or systemic sclerosis in a subject. In some embodiments, the subject is a subject having, suspected of having, or at risk of developing a disorder associated with fibrosis. In some embodiments, the subject is a subject having, suspected of having, or at risk of developing systemic sclerosis. In some embodiments, the subject is a mammalian subject, including but not limited to a dog, cat, horse, cow, pig, sheep, goat, rodent, or primate. In some embodiments, the subject is a human subject, such as a human patient. The human subject may be a pediatric or adult subject. Whether a subject is deemed “at risk” of having fibrosis or systemic sclerosis may be determined by a skilled practitioner. In some embodiments, the subject has been diagnosed with a disorder associated with fibrosis. In some embodiments, the subject has been diagnosed with a disorder associated with systemic sclerosis. In some embodiments, the subject is a human that presents one or more symptoms associated with a disorder associated with fibrosis or system sclerosis.

### Therapeutically Effective Amount

In one aspect, the disclosure provides methods of treating a disorder associated with fibrosis or systemic sclerosis with a therapeutically effective amount of an agent that modulates a protein provided in Table 1. As used herein, a “therapeutically effective amount” and “effective amount,” which are used interchangeably herein, refer to an amount of an agent that is sufficient to improve or enhance at least one aspect of the disease or disorder. In some embodiments, the therapeutically effective amount is an amount that reduces one or more symptoms of the disease or disorder, and/or enhances the survival of the subject having the disease or disorder. In some embodiments, the subject is administered a therapeutically effective amount of the agent to reduce production or accumulation of collagen. In some embodiments, the subject is administered a therapeutically effective amount of the modulator to reduce inflammation.

In some embodiments, the therapeutically effective amount of an agent is an amount effective in preventing or delaying the onset of a disorder associated with fibrosis or systemic sclerosis or one or more symptoms thereof.

Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be selected which does not cause substantial toxicity and yet is effective to treat the particular subject.

The therapeutically effective amount of an agent can vary depending on such factors as the disorder or condition being treated, the particular agent(s) to be administered and properties thereof, the size of the subject, the gender of the subject, or the severity of the disorder. One of ordinary skill in the art can empirically determine the therapeutically effective amount of an agent without necessitating undue experimentation. In some embodiments, it is preferred that a maximum dose be used, that is, the highest safe dose according to some medical judgment. Multiple doses per day, week or month may be contemplated to achieve appropriate levels of the agent (e.g., systemic levels and/or local levels). In some embodiments, the agent that modulates the expression and/or activity of any one or more of the proteins provided in Table 1 is administered in a single dose. In some embodiments, the agent that modulates the expression and/or activity of any one or more of the proteins provided in Table 1 is administered in multiple doses, such as multiple doses administered concomitantly or sequentially. In some embodiments, 1, 2, 3, 4, 5, 6, 7, 8, 9, 10, or more than 10 doses of the agent are administered. In some embodiments, one or more loading doses of the agent is administered, following by one or more maintenance doses of the agent. In some embodiments, doses are administered at regular intervals while in other embodiments doses are administered at irregular intervals. In some embodiments, the agent is administered for an indefinite period of time. Appropriate systemic levels of the agent can be determined by, for example, quantification of the agent in a blood or serum sample from the subject, assessing expression and/or activity of the protein modulated by the agent. Any of the methods of administration can include monitoring levels of the agent, monitoring activity and/or expression, assessing any one or more symptoms of the disorder, and dose adjustment as needed.

In some embodiments, the agent is administered with one or more additional agents, such as therapeutic agents. The additional agents can be administered before, simultaneously, or after administration of the agent. In some embodiments, an additional agent is an antibody or fragment thereof, a protein, a fusion protein, a small molecule, or a nucleic acid. In some embodiments, 2, 3, 4, 5, or more than 5 additional agents are administered.

In some embodiments, more than one agent that modulates the expression and/or activity of any of the proteins provided in Table 1 are administered to the subject. In some embodiments, at least 2, 3, 4, 5, or more agents that modulate the expression and/or activity of any of the proteins provided in Table 1 are administered to the subject. In some embodiments, the more than one agents are administered to the subject at the same time, for example in a combined dose.

In some embodiments, when more than one agent is administered to the subject at different times, for example a first agent is administered to the subject and a second agent is administered to the subject at a subsequent time. In some embodiments, the amount of a therapeutically effective amount of an agent administered in combination with one or more additional agents is less than the therapeutically effective amount of the agent when administered in the absence of an additional agent.

In methods for treating a disorder related to fibrosis or systemic sclerosis in a subject, a therapeutically effective amount of an agent is any amount that provides an anti-fibrotic effect, such as reduces or prevents production or accumulation of extracellular matrix proteins. In some embodiments, the therapeutically effective amount of an agent that modulates expression and/or activity of any of the proteins described herein is reduced when the agent is administered concomitantly or sequentially with any one or more additional agents of the proteins as compared to the effective amount of the agent when administered in the absence of the additional agent(s). In some embodiments, the effective amount of an agent that modulates expression and/or activity of any of the proteins provided in Table 1 is reduced by at least 1.1-, 1.2-, 1.3-, 1.4-, 1.5-, 1.6-, 1.7-, 1.8-, 1.9-, 2.0-, 2.1-, 2.2-, 2.3-, 2.4-, 2.5-, 2.6-, 2.7-, 2.8-, 2.9-, 3.0-, 4.0-, 5.0-, 10.0-, 15.0-, 20.0-, 25.0-, 30.0-, 35.0-, 40.0-, 45.0-, 50.0-, 55.0-, 60.0-, 65.0-, 70.0-, 75.0-, 80.0-, 85.0-, 90.0-, 95.0-, 100-, 200-, 300-, 400-, or at least 500-fold or more when the agent is concomitantly or sequentially administered with one or more additional agents (e.g., combinations of two agents that modulate expression and/or activity of the same or different target proteins presented in Table 1).

In some embodiments, the therapeutically effective amount of an agent is an amount sufficient to reduce fibrosis by at least 10%, at least 20%, at least 30%, at least 40% at least 50%, at least 60%, at least 70%, at least 80%, at least 90%, at least 95%, at least 96%, at least 97%, at least 98%, or at least 99% compared to fibrosis in the absence of the agent. In some embodiments, the therapeutically effective amount of an agent is an amount sufficient to reduce the severity of one or more symptoms of the disorder by at least 10%, at least 20%, at least 30%, at least 40% at least 50%, at least 60%, at least 70%, at least 80%, at least 90%, at least 95%, at least 96%, at least 97%, at least 98%, or at least 99% compared to the severity of the symptom in the absence of the agent.

In some embodiments, the therapeutically effective amount of an agent that modulates the expression and/or activity of a protein provided in Table 1 is an amount sufficient to reduce inflammation. In some embodiments, the therapeutically effective amount of an agent is an amount sufficient to reduce the quantity of pro-inflammatory or inflammatory factors, for example pro-inflammatory or inflammatory cytokines (e.g., interleukin 1 (IL-1), interleukin 6 (IL-6), TNFα). In some embodiments, the therapeutically effective amount of an agent is an amount sufficient to reduce the quantity of IL-1 and/or IL-6 and/or TNFα by at least 10%, at least 20%, at least 30%, at least 40% at least 50%, at least 60%, at least 70%, at least 80%, at least 90%, at least 95%, at least 96%, at least 97%, at least 98%, or at least 99%, as compared to the quantity of IL-1 and/or IL-6 and/or TNFα in a subject (systemically or locally) prior to or in the absence of administration of the agent.

### Administration

Aspects of the disclosure provide methods for treating disorders associated with fibrosis and systemic sclerosis in a subject comprising administering to the subject an agent that modulates the expression and/or activity of any one or more of the proteins provided in Table 1. As used herein “treating” can include: improving one or more symptoms of a disorder; curing a disorder; preventing a disorder from becoming worse; slowing the rate of progression of a disorder; or preventing a disorder from re-occurring (e.g., preventing a relapse).

In some embodiments, the agent is administered orally, parenterally, intravenously, topically, subcutaneously, or by inhalation. In some embodiments, the agent is administered by continuous infusion. Selection of an appropriate route of administration will depend on various factors not limited to the particular disorder and/or severity of the disorder.

In some embodiments, the agent is administered in one dose. In some embodiments, the agent is administered in multiple doses. In some embodiments, more than one agent (e.g., 2, 3, 4, 5, or more agents) are administered together in one dose. In some embodiments, more than one agent (e.g., 2, 3, 4, 5, or more agents) are administered in separate doses. In some embodiments, the multiple or separate doses are administered by the same route of administration (e.g., each dose is administered intravenously). In some embodiments, the multiple or separate doses are administered by different routes of administrations (e.g., one dose is administered intravenously and another dose(s) is administered orally).

Any agent that modulates expression and/or activity of any one or more of the proteins provided in Table 1 can be administered to a subject as a pharmaceutical composition, which may routinely contain pharmaceutically acceptable concentrations of salt, buffering agents, preservatives, compatible carriers, adjuvants, pharmaceutically acceptable excipients, and optionally other therapeutic ingredients. The nature of the pharmaceutical carrier, excipient, and other components of the pharmaceutical composition will depend on the mode of administration. The pharmaceutical compositions of the disclosure may be administered by any means and route known to the skilled artisan in carrying out the treatment methods described herein.

Any of the agents described herein, that modulates expression and/or activity of a protein provided in Table 1 may be delivered systemically. In some embodiments, the agent is formulated for parenteral administration by injection. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In some embodiments, the agent is formulated for oral administration. In some embodiments, the agent is formulated readily by combining the compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral administration can be obtained as solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers, e.g., EDTA for neutralizing internal acid conditions, or may be administered without any carriers.

For oral delivery, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on Tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

Any of the agents described herein may be provided in the formulation as fine multi-particulates in the form of granules or pellets. The formulation of the material for capsule administration could also be as a powder, lightly compressed plugs or even as tablets. The pharmaceutical composition could be prepared by compression. One may dilute or increase the volume of the pharmaceutical composition with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the pharmaceutical composition, such as in a solid dosage form. Materials used as disintegrants include but are not limited to starch, including the commercial disintegrant based on starch, Explotab®, sodium starch glycolate, Amberlite, sodium carboxymethylcellulose, ultramylopectin, sodium alginate, gelatin, orange peel, acid carboxymethyl cellulose, natural sponge and bentonite may also be used. Binders may be used to hold the therapeutic agent together to form a hard tablet and include materials from natural products such as acacia, tragacanth, starch and gelatin. An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Glidants that might improve the flow properties of the drug during formulation and to aid rearrangement during compression might be added. The glidants may include starch, talc, pyrogenic silica and hydrated silicoaluminate.

For administration by inhalation, the agent may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas.

Also contemplated herein is pulmonary delivery of an agent that modulates expression and/or activity of one or more proteins provided in Table 1. The agent may be delivered to the lungs of a mammal for local or systemic delivery. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13:143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al. Nasal delivery of a pharmaceutical composition comprising an agent that modulates the expression and/or activity of a protein of Table 1 is also contemplated. Nasal delivery allows the passage of a pharmaceutical composition to the blood stream directly after administering the composition to the nose, without the necessity for deposition of the product in the lung.

In some embodiments, the agent is administered locally. Local administration methods are known in the art and will depend on the target area or target organ. Local administration routes include the use of standard topical administration methods such as epicutaneous (application onto the skin), by inhalational, rectal (e.g., by enema or suppository), by eye drops (onto the conjunctiva), ear drops, intranasal route, and vaginal.

The agents may also be formulated in rectal or vaginal compositions such as suppositories or retention enemas, e.g., containing conventional suppository bases such as cocoa butter or other glycerides. In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble analogs, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose analogs, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or one or more auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference. The agents and compositions described herein may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

The pharmaceutical compositions of the disclosure contain an effective amount of an agent with a pharmaceutically-acceptable carrier or excipient. The term pharmaceutically acceptable excipient means one or more compatible solid or liquid filler, diluents or encapsulating substances which are suitable for administration to a human or other vertebrate animal. The term excipient denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

Both non-biodegradable and biodegradable polymeric materials can be used in the manufacture of particles for delivering the compositions of the disclosure. Such polymers may be natural or synthetic polymers. The polymer is selected based on the period of time over which release is desired. Bioadhesive polymers of particular interest include bioerodible hydrogels described by Sawhney et al., 1993, Macromolecules 26, 581-587, the teachings of which are incorporated herein by reference. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(5 octadecyl acrylate).

The agents described herein may be contained in controlled release systems. The term “controlled release” is intended to refer to any agents and compositions described herein containing formulation in which the manner and profile of agents and compositions described herein release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a compound over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the compound therefrom. “Delayed release” may or may not involve gradual release of a compound over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless required by context, singular terms shall include pluralities and plural terms shall include the singular. The methods and techniques of the present disclosure are generally performed according to conventional methods well-known in the art. Generally, nomenclature used in connection with, and techniques of biochemistry, enzymology, molecular and cellular biology, microbiology, genetics and protein and nucleic acid chemistry and hybridization described herein are those well-known and commonly used in the art. The methods and techniques of the present disclosure are generally performed according to conventional methods known in the art and as described in various general and more specific references that are cited and discussed throughout the present specification unless otherwise indicated. The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Protein Identification

To identify potential upstream regulators involved in aspects of disorders associated with fibrosis, gene expression data comparing systemic sclerosis skin samples to healthy skin samples and protein interaction networks were analyzed. In some embodiments, genes/proteins that were or were not differentially expressed in the systemic sclerosis skin samples but had a significant number of known interactions with differentially expressed genes/proteins were selected.

First, the protein interaction network was reduced to only include genes represented in the gene expression data set. The protein interaction network was also reduced to only include interactions which have a score of at least 500 in the STRING network (Franceschini et al. Nucleic Acids Res. (2013) 41: D808-D815; von Mering et al. Nucleic Acids Res. (2005) 33: D433-7). Genes were scored base on the differential expression between the systemic sclerosis skin samples and healthy skin samples using a two-sided and unequal variances t-test.

Genes were each scored for their “attachment” (number of known interactions in the STRING network) to differentially expressed genes. A gene/protein was first scored for its number of interactions with the most differentially expressed gene (smallest FDR value). This yielded an edge-count probability “Pa1,” which takes into account total known numbers of interactions for both the differentially expressed and the non-differentially expressed genes (Pradines et al. J. Comput. Biol. (2005) 12(2): 113-28). The gene was then scored for the number of interactions with the two most differentially expressed genes. This yielded an edge-count probability referred to as “Pa2.”

This analysis process was repeated for all possible n ordered sets of differentially expressed genes. The gene/protein was then given the score minPa=min(Pa1, Pa2, . . . , Pan), between 0 and 1. Values of minPa close to 0 indicate the proteins had a significant number of interactions with differentially expressed genes. Computation of all Pa values was performed using techniques described in Pradines et al. Research in Computational Molecular Biology 2007: 296-310.

Values of minPa obtained using two gene expression data sets (GSE58095 (Assassi et al. Arthritis Rheumatol. (2015) 67(11): 3016-26; www.ncbi.nlm.nih.gov/geo/query/acc.cgi?acc=GSE58095) and GSE32413 (Pendergrass et al. J. Invest. Dermatol. (2012) 132(5): 1363-73; www.ncbi.nlm.nih.gov/geo/query/acc.cgi?acc=GSE32413) were combined. Each protein was scored with the maximum value of its two minPa values, resulting in maxminPa. To correct for multiple-testing, the FDR method was utilized on the entire vector of maxminPa values. This resulted in a final score: a p-value between 0 and 1. Small p-value, i.e. values close to 0, indicate proteins that were not differentially expressed in either data set but have a significantly large number of interactions with differentially expressed genes in both data sets.

Results from this analysis are presented in Table 1.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

The foregoing written specification is considered to be sufficient to enable one skilled in the art to practice the invention. The present invention is not to be limited in scope by the examples provided, since the examples are intended as an illustration of certain aspects and embodiments of the invention. Other functionally equivalent embodiments are within the scope of the invention.

Those skilled in the art will recognize, or be able to ascertain using no more than routine experimentation, many equivalents of the invention described herein. Such equivalents are intended to be encompassed by the following claims. All references, including patent documents, disclosed herein are incorporated by reference in their entirety.

1. A method for treating a disorder associated with fibrosis, the method comprising
administering to a subject having a disorder associated with fibrosis a therapeutically effective amount of an agent that modulates the expression and/or activity of any one or more of the proteins set forth in Table 1.

2. The method of claim 1, wherein the agent inhibits the expression and/or activity of any one or more of the proteins set forth in Table 1.

3. The method of claim 1, wherein the agent enhances the expression and/or activity of any one or more of the proteins set forth in Table 1.

4. The method of claim 1, wherein the agent is an antibody or fragment thereof, a protein, a fusion protein, a small molecule, or a nucleic acid.

5. The method of claim 4, wherein the agent is an antibody that selectively binds to any one or more of the proteins set forth in Table 1.

6. The method of claim 1, wherein the agent modulates expression of a nucleic acid encoding any one or more of the proteins set forth in Table 1.

7. The method of claim 1, wherein the agent is selected from any of the agents set forth in Table 3.

8.-12. (canceled)

13. The method of claim 1, wherein the disorder associated with fibrosis is pulmonary fibrosis, cirrhosis, atrial fibrosis, endomyocardial fibrosis, glial scar, arthrofibrosis, Crohn's disease, Dupuytren's contracture, keloid, mediastinal fibrosis, 10 myelofibrosis, Peyronie's disease, nephrogenic systemic fibrosis, progressive massive fibrosis, retroperitoneal fibrosis, systemic sclerosis, skeletal muscle fibrosis, or adhesive capsulitis.

14.-15. (canceled)

16. A method for treating systemic sclerosis, the method comprising
administering to a subject having systemic sclerosis a therapeutically effective amount of an agent that modulates the expression and/or activity of any one or more of the proteins set forth in Table 1.

17. The method of claim 16, wherein the systemic sclerosis is limited cutaneous systemic sclerosis or diffuse cutaneous systemic sclerosis.

18. The method of claim 16, wherein the agent inhibits the expression and/or activity of any one or more of the proteins set forth in Table 1.

19. The method of claim 16, wherein the agent enhances the expression and/or activity of any one or more of the proteins set forth in Table 1.

20. The method of claim 16, wherein the agent is an antibody or fragment thereof, a protein, a fusion protein, a small molecule, or a nucleic acid.

21. The method of claim 20, wherein the agent is an antibody that selectively binds to any one or more of the proteins set forth in Table 1.

22. The method of claim 16, wherein the agent modulates expression of a nucleic acid encoding any one or more of the proteins set forth in Table 1.

23. The method of claim 16, wherein the agent is selected from any of the agents set forth in Table 3.

24.-30. (canceled)
